(19) United States
(12) Patent Application Publication
(10) Pub. No.: US 2026/0027325 A1
(43) Pub. Date: Jan. 29, 2026

(54) TUBE, METHOD FOR MANUFACTURING TUBE, CATHETER, AND MEDICAL TUBULAR OBJECT DELIVERY DEVICE

(71) Applicant: KANEKA CORPORATION, Osaka-shi, Osaka (JP)

(72) Inventors: Kentaro SUZUKI, Settsu-shi (JP); Mei FUKAMI, Settsu-shi (JP)

(73) Assignee: KANEKA CORPORATION, Osaka-shi, Osaka (JP)

(21) Appl. No.: 18/846,332

(22) PCT Filed: Mar. 9, 2023
(86) PCT No.: PCT/JP2023/009170

§ 371 Date: Mar. 28, 2025

(30) Foreign Application Priority Data
Mar. 17, 2022 (JP) 2022-042890

(51) Int. Cl. A61M 25/00; A61F 2/962; A61F 2/966

(52) U.S. Cl. CPC A61M 25/0045; A61F 2/966; A61M 25/0012; A61M 25/0052; A61F 2002/9623; A61M 2207/00

(57) ABSTRACT

A method for manufacturing a tube, comprising the steps of: disposing a first inner cylindrical body (11) and a second inner cylindrical body (12) side by side in a longitudinal direction; disposing a reinforcing layer (13) composed of wire from an outer side of the first inner cylindrical body (11) to an outer side of the second inner cylindrical body (12); disposing an outer layer (14) on an outer side of the reinforcing layer (13) to obtain a tube precursor (15); and heating the tube precursor (15); wherein: a melting point or decomposition point of a resin constituting the first inner cylindrical body (11) is higher than a melting point of a resin constituting the second inner cylindrical body (12) and a melting point of a resin constituting the outer layer (14); and a heating temperature in the step of heating the tube precursor (15) is equal to or higher than the melting point of the resin constituting the second inner cylindrical body (12) and the melting point of the resin constituting the outer layer (14), and lower than the melting point or decomposition point of the resin constituting the first inner cylindrical body (11).

### TECHNICAL FIELD

The present invention relates to a tube having a reinforcing layer composed of wire, a method for manufacturing the tube, and a catheter and a medical tubular object delivery device provided with the tube.

### BACKGROUND ART

Conventionally, tubes having a reinforcing layer composed of wire have been known, and used for various applications. Tubes having a reinforcing layer is usually configured so that the reinforcing layer is disposed between an inner layer and an outer layer, and have excellent tensile strength and bending strength and are difficult to break. As a method for manufacturing a tube having a reinforcing layer, for example, Patent Literature 1 discloses a method for manufacturing a tube comprising the steps of: coating a first thermoplastic resin on a continuous core rod; disposing a braid on the first thermoplastic resin; removing the braid and the first thermoplastic resin at constant intervals from the surface of the continuous core rod; coating a second thermoplastic resin on a portion where the braid is disposed and a portion where the braid and the first thermoplastic resin are removed and cutting at a certain position; and pulling out the continuous core rod.

### CITATION LISTS

### Patent Literatures

### Patent Literature 1

Japanese Unexamined Laid-open Patent Application Publication No. H11-245311

### SUMMARY OF INVENTION

### Technical Problem

Since a tube having a reinforcing layer composed of wire has high breaking strength and excellent reliability, it is used for medical devices such as catheters. In such medical devices, there are cases that another device may be inserted in a lumen of the tube or placed therein. In such cases, the tube is required to have high slipperiness on the inner surface thereof, and is required so that its inner surface has sufficient strength without being damaged. Meanwhile, it is also required that the tube as a whole, including the inner layer, the outer layer and the reinforcing layer, has integrity.

The present invention has been made in view of the above circumferences, and an object of the present invention is to provide a tube having a reinforcing layer composed of wire, in which the inner layer disposed on the inner side of the reinforcing layer can be made of various resins, and the integrity as the tube is easily ensured, and a method for manufacturing the tube. The present invention also provides a catheter and a medical tubular object delivery device each comprising the tube of the present invention.

### Solution to Problem

A method for manufacturing a tube of the present invention that can solve the above problem is as follows.
[1] A method for manufacturing a tube extending in a longitudinal direction and having a reinforcing layer composed of wire, comprising the steps of:
disposing a first inner cylindrical body and a second inner cylindrical body side by side in the longitudinal direction;
disposing a reinforcing layer composed of wire from an outer side of the first inner cylindrical body to an outer side of the second inner cylindrical body;
disposing an outer layer on an outer side of the reinforcing layer to obtain a tube precursor; and
heating the tube precursor;
wherein:
a melting point or decomposition point of a resin constituting the first inner cylindrical body is higher than a melting point of a resin constituting the second inner cylindrical body and a melting point of a resin constituting the outer layer; and
a heating temperature in the step of heating the tube precursor is equal to or higher than the melting point of the resin constituting the second inner cylindrical body and the melting point of the resin constituting the outer layer, and lower than the melting point or decomposition point of the resin constituting the first inner cylindrical body.

According to the method for manufacturing a tube of the present invention, a tube with excellent integrity can be obtained by disposing the second inner cylindrical body made of a resin having a relatively low melting point side by side in the longitudinal direction with the first inner cylindrical body made of a resin having a relatively high melting point or high decomposition point resin, disposing the outer layer made of a resin having a relatively low melting point on the outer side of the first inner cylindrical body and the second inner cylindrical body with the reinforcing layer interposed therebetween, and heating them. Furthermore, since the first inner cylindrical body is made of a resin having a relatively high melting point or high decomposition point, the variety of resins that can be used for the first inner cylindrical body increases, and it becomes easy to impart desired characteristics to the first inner cylindrical body.

Preferred embodiments of the method for manufacturing a tube of the present invention are as follows.
[2] The method for manufacturing a tube according to [1] above, wherein the first inner cylindrical body is made of a fluororesin, a polyimide resin, a polyamideimide resin or an aromatic polyether ketone resin.
[3] The method for manufacturing a tube according to [1] or [2] above, wherein the second inner cylindrical body and the outer layer are each independently made of a polyester resin, a polyamide resin, a polyolefin resin, a polyurethane resin or a polyether polyamide resin.
[4] The method for manufacturing a tube according to any one of [1] to [3] above, wherein the second inner cylindrical body and the outer layer are made of a same resin.
[5] The method for manufacturing a tube according to any one of [1] to [4] above, wherein the tube precursor has a section including the first inner cylindrical body, the reinforcing layer and the outer layer, a section including the second inner cylindrical body, the reinforcing layer and the outer layer, and a section including the second inner cylindrical body and the outer layer but not including the reinforcing layer, in this order.

In the above case, the second inner cylindrical body extends from the portion where it overlaps with the reinforcing layer to the portion where it does not overlap with the reinforcing layer, and the outer layer also extends from the portion where it overlaps with the reinforcing layer to the portion where it does not overlap with the reinforcing layer. Therefore, by heating the tube precursor in the heating step, the second inner cylindrical body and the outer layer can be joined to each other more firmly in the section where the reinforcing layer is not present.

A tube of the present invention that can solve the above problem is as follows.
[6] A tube extending in a longitudinal direction, comprising an inner layer, an outer layer and a reinforcing layer composed of wire disposed between the inner layer and the outer layer, wherein:
the inner layer includes a first inner layer and a second inner layer disposed side by side in the longitudinal direction;
the reinforcing layer is disposed from an outer side of the first inner layer to an outer side of the second inner layer; and
a melting point or decomposition point of a resin constituting the first inner layer is higher than a melting point of a resin constituting the second inner layer and a melting point of a resin constituting an outer layer.

According to the tube of the present invention, desired characteristics, derived from the first inner layer, can be imparted to the inner layer disposed on the inner side of the reinforcing layer. Since the first inner layer is made of a resin with a relatively high melting point or high decomposition point, the variety of resins that can be used for the first inner layer increases, and it is easy to impart desired characteristics to the first inner layer. In addition, since the second inner layer is disposed adjacent to the first inner layer in the longitudinal direction and the second inner layer and the outer layer are made of a resin with a relatively low melting point, the second inner layer, the reinforcing layer and the outer layer can be integrated by welding. Furthermore, in the tube, a boundary between the first inner layer and the second inner layer is located at a position where it overlaps with the reinforcing layer, and therefore, the boundary between the first inner layer and the second inner layer is reinforced by the reinforcing layer, and the tube is less likely to break at the boundary between the first inner layer and the second inner layer when using the tube.

Preferred embodiments of the tube of the present invention are as follows.
[7] The tube according to [6] above, wherein the first inner layer is made of a fluororesin, a polyimide resin, a polyamideimide resin or an aromatic polyether ketone resin.
[8] The tube according to [6] or [7] above, wherein the second inner layer and the outer layer are each independently made of a polyester resin, a polyamide resin, a polyolefin resin, a polyurethane resin or a polyether polyamide resin.
[9] The tube according to any one of [6] to [8] above, wherein the second inner layer and the outer layer are made of a same resin.
[10] The tube according to any one of [6] to [9] above, wherein the tube has a section including the first inner layer, the reinforcing layer and the outer layer, a section including the second inner layer, the reinforcing layer and the outer layer, and a section including the second inner layer and the outer layer but not including the reinforcing layer, in this order.

In the tube configured as described above, the second inner layer and the outer layer are joined to each other more firmly, and the tube is less prone to breakage or kinking at the end of the reinforcing layer. Therefore, the reliability of the tube can be improved.

A catheter of the present invention is as follows.
[11] A catheter comprising the tube according to any one of [6] to [10] above.

By applying the tube of the present invention to a catheter, a catheter with excellent reliability can be obtained.

A medical tubular object delivery device of the present invention is as follows.
[12] A medical tubular object delivery device comprising the tube according to any one of [6] to [10] above, wherein: the tube has a first section including a distal end and a second section proximal to the first section in the longitudinal direction; the first inner layer is provided at the first section and the second inner layer is provided at the second section; and a medical tubular object is placed in a lumen of the first section of the tube.

By applying the tube of the present invention to a medical tubular object delivery device, the stable operation of implanting the medical tubular object in the body is facilitated.

### Advantageous Effects of Invention

According to the method for manufacturing a tube of the present invention and the tube of the present invention, the inner layer disposed on the inner side of the reinforcing layer can be made of various resins, which makes it easier to desired characteristics to the inner layer disposed on the inner side of the reinforcing layer, and the integrity of the tube can be ensured. In addition, a catheter and a medical tubular object delivery device provided with the tube of the present invention have excellent operability and reliability.

### BRIEF DESCRIPTION OF DRAWINGS

FIG. 1 shows an outline of the entire process of a method for manufacturing a tube according to an embodiment of the present invention.

FIG. 2 represents a configuration example of a tube according to an embodiment of the present invention, and shows a cross-sectional view along a longitudinal direction of the tube.

FIG. 3 represents a configuration example of a tube according to an embodiment of the present invention, and shows a cross-sectional view along a longitudinal direction of the tube.

FIG. 4 represents a configuration example of a tube according to an embodiment of the present invention, and shows a cross-sectional view along a longitudinal direction of the tube.

FIG. 5 represents a configuration example of a tube according to an embodiment of the present invention, and shows a cross-sectional view along a longitudinal direction of the tube.

FIG. 6 shows an overall schematic view of a medical tubular object delivery device according to an embodiment of the present invention.

FIG. 7 shows an example of the configuration of a shaft of a medical tubular object delivery device.

FIG. 8 shows an example of the configuration of a shaft of a medical tubular object delivery device.

FIG. 9 shows an example of the configuration of a shaft of a medical tubular object delivery device.

FIG. 10 shows an example of the configuration of a shaft of a medical tubular object delivery device.

### DESCRIPTION OF EMBODIMENTS

Hereinafter, the present invention is specifically explained below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching or a reference sign for a member may be omitted for convenience, and in such a case, the description and other drawings should be referred to. In addition, sizes of various members in the drawings may differ from the actual sizes thereof, since priority is given to understanding the features of the present invention.

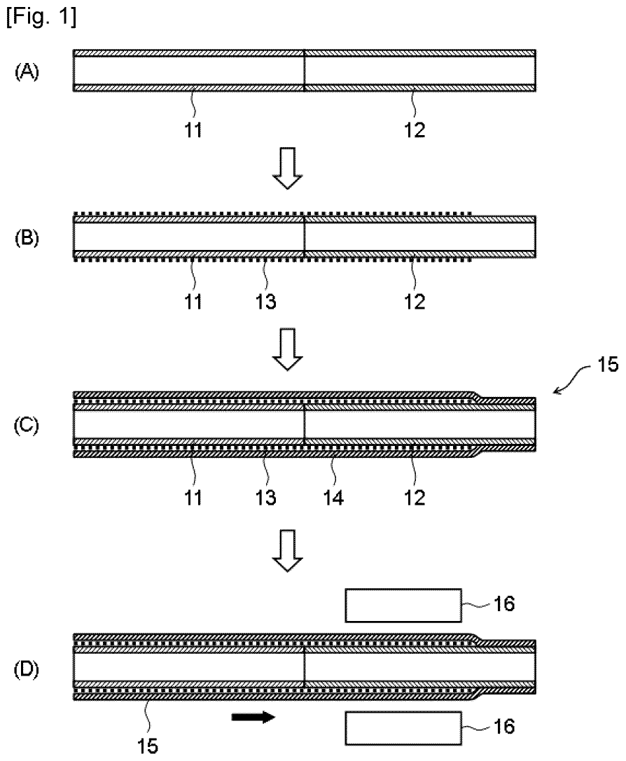

First, a method for manufacturing a tube of the present invention is explained with reference to FIG. 1. FIG. 1 shows an outline of the entire process of a method for manufacturing a tube according to an embodiment of the present invention. FIG. 1 shows a sectional view along a longitudinal direction of the tube.

A method for manufacturing a tube according to an embodiment of the present invention is a method for manufacturing a tube extending in a longitudinal direction and having a reinforcing layer composed of wire, comprising the steps of: disposing a first inner cylindrical body and a second inner cylindrical body side by side in the longitudinal direction (hereinafter may be referred to as an “inner cylindrical body arrangement step”); disposing a reinforcing layer composed of wire from an outer side of the first inner cylindrical body to an outer side of the second inner cylindrical body (hereinafter may be referred to as a “reinforcing layer arrangement step”); disposing an outer layer on an outer side of the reinforcing layer to obtain a tube precursor (hereinafter may be referred to as an “outer layer arrangement step”); and heating the tube precursor (hereinafter may be referred to as a “heating step”); wherein: a melting point or decomposition point of a resin constituting the first inner cylindrical body is higher than a melting point of a resin constituting the second inner cylindrical body and a melting point of a resin constituting the outer layer; and a heating temperature in the step of heating the tube precursor is equal to or higher than the melting point of the resin constituting the second inner cylindrical body and the melting point of the resin constituting the outer layer, and lower than the melting point or decomposition point of the resin constituting the first inner cylindrical body. In FIG. 1, the inner cylindrical body arrangement step is shown in FIG. 1(A), the reinforcing layer arrangement step is shown in FIG. 1(B), the outer layer arrangement step is shown in FIG. 1(C), and the heating step is shown in FIG. 1(D).

In the inner cylindrical body arrangement step, a first inner cylindrical body 11 and a second inner cylindrical body 12 are disposed side by side in the longitudinal direction. The longitudinal direction refers to a direction in which the tube extends, and the tube has a lumen extending in the longitudinal direction. The first inner cylindrical body 11 and the second inner cylindrical body 12 form an inner layer of the tube. From the first inner cylindrical body 11, a first inner layer is formed, and from the second inner cylindrical body 12, a second inner layer is formed. The first inner cylindrical body 11 and the second inner cylindrical body 12 are disposed so as to be in contact with each other in the longitudinal direction. Each of the first inner cylindrical body 11 and the second inner cylindrical body 12 has a lumen extending in the longitudinal direction, and by disposing the first inner cylindrical body 11 and the second inner cylindrical body 12 side by side in the longitudinal direction, a lumen extending from the first inner cylindrical body 11 to the second inner cylindrical body 12 is formed.

The first inner cylindrical body 11 and the second inner cylindrical body 12 are made of resin. The type of resin constituting the first inner cylindrical body 11 and the second inner cylindrical body 12 is not particularly limited, but the second inner cylindrical body 12 is preferably made of thermoplastic resin. The first inner cylindrical body 11 may be made of thermoplastic resin or thermosetting resin. In the case where the first inner cylindrical body 11 or the second inner cylindrical body 12 is made of thermoplastic resin, the resin forming the first inner cylindrical body 11 or the second inner cylindrical body 12 has a melting point. In the case where the first inner cylindrical body 11 is made of thermosetting resin, the resin forming the first inner cylindrical body 11 has a decomposition point.

A melting point or decomposition point of the resin constituting the first inner cylindrical body 11 is higher than a melting point of the resin constituting the second inner cylindrical body 12. By forming the first inner cylindrical body 11 from a resin with a relatively high melting point or high decomposition point, the variety of resins that can be used for the first inner cylindrical body 11 increases, thereby making it easier to give the first inner cylindrical body 11 desired characteristics. Meanwhile, by forming the second inner cylindrical body 12 from a resin with a relatively low melting point, it becomes easy to melt and bond the second inner cylindrical body 12 to the outer layer 14 in the later heating step.

The difference between the melting point or decomposition point of the resin constituting the first inner cylindrical body 11 and the melting point of the resin constituting the second inner cylindrical body 12 is preferably 30° C. or higher, more preferably 40° C. or higher, and even more preferably 50° C. or higher. The upper limit of the difference between the melting point or decomposition point of the resin constituting the first inner cylindrical body 11 and the melting point of the resin constituting the second inner cylindrical body 12 is not particularly limited, and may be, for example, 300° C. or lower, 250° C. or lower, 200° C. or lower, or 150° C. or lower.

Examples of the resin constituting the first inner cylindrical body 11 and the second inner cylindrical body 12 include synthetic resins such as polyolefin resin (e.g., polyethylene and polypropylene), polyamide resin (e.g., nylon), polyester resin (e.g., polyethylene terephthalate), aromatic polyether ketone resin (e.g., PEEK), polyether polyamide resin, polyurethane resin, polyimide resin, polyamideimide resin, fluororesin (e.g., PTFE, PFA and ETFE), polyvinyl chloride resin, silicone resin, and others.

The first inner cylindrical body 11 is preferably made of a fluororesin, a polyimide resin, a polyamideimide resin or an aromatic polyetherketone resin, as a resin having a relatively high melting point or high decomposition point. By forming the first inner cylindrical body 11 from such a resin, the inner surface of the tube formed from the first inner cylindrical body 11 can be made to have increased slipperiness and the inner surface of the tube can be made less susceptible to damage and have sufficient strength.

The second inner cylindrical body 12 is preferably made of a polyester resin, a polyamide resin, a polyolefin resin, a polyurethane resin or a polyether polyamide resin, as a resin having a relatively low melting point. By forming the second inner cylindrical body 12 from such a resin, it becomes easy to melt and bond the second inner cylindrical body 12 to the outer layer 14 in the later heating step.

The inner diameter of the first inner cylindrical body 11 and the inner diameter of the second inner cylindrical body 12 are preferably approximately the same. The outer diameter of the first inner cylindrical body 11 and the outer diameter of the second inner cylindrical body 12 are preferably approximately the same. The inner diameters of the first inner cylindrical body 11 and the second inner cylindrical body 12 are not particularly limited, and in the case where the tube is applied to a catheter or a medical tubular object delivery device, the inner diameters of the first inner cylindrical body 11 and the second inner cylindrical body 12 may be, for example, about 0.3 mm to 3.5 mm. The thickness of the first inner cylindrical body 11 and the second inner cylindrical body 12 may be, for example, about 0.01 mm to 0.3 mm.

In the inner cylindrical body arrangement step, it is preferable that a rod is prepared and inserted into the lumen of the first inner cylindrical body 11 and the lumen the second inner cylindrical body 12, and in this state, the first inner cylindrical body 11 and the second inner cylindrical body 12 are disposed side by side in the longitudinal direction. Thereby, the first inner cylindrical body 11 and the second inner cylindrical body 12 can be stably arranged side by side in the longitudinal direction. As the rod, a metal rod or the like can be used.

In the reinforcing layer arrangement step, a reinforcing layer 13 composed of wire is disposed from an outer side of the first inner cylindrical body 11 to an outer side of the second inner cylindrical body 12. The reinforcing layer 13 is disposed on the outer sides of the first inner cylindrical body 11 and the second inner cylindrical body 12 so as to cover both the first inner cylindrical body 11 and the second inner cylindrical body 12. The reinforcing layer 13 forms a reinforcing layer of the tube. The reinforcing layer 13 is preferably disposed so as to surround the outer side of the first inner cylindrical body 11 and the outer side of the second inner cylindrical body 12. By disposing the reinforcing layer 13 from the outer side of the first inner cylindrical body 11 to the outer side of the second inner cylindrical body 12, the resulting tube is reinforced by the reinforcing layer 13 at a boundary between the first inner cylindrical body 11 and the second inner cylindrical body 12, and becomes less likely to break at the boundary between the first inner cylindrical body 11 and the second inner cylindrical body 12.

The reinforcing layer 13 disposed from the outer side of the first inner cylindrical body 11 to the outer side of the second inner cylindrical body 12 may have a cylindrical shape or a sheet shape. In the former case, the reinforcing layer 13 of a cylindrical shape is disposed outside the first inner cylindrical body 11 and the second inner cylindrical body 12. In the latter case, it is preferable that the reinforcing layer 13 of a sheet shape is wound around the outside of the first inner cylindrical body 11 and the second inner cylindrical body 12.

Examples of the wire that constitutes the reinforcing layer 13 include metal wires, fibers, and others. Examples of material constituting the metal wire include stainless steel such as SUS304 and SUS316, carbon steel, platinum, nickel, cobalt, chromium, titanium, tungsten, gold, nickel-titanium alloy, cobalt-chromium alloy, tungsten alloy, and others; and among them, stainless steel is preferable. The metal wire may be a single wire or a twisted wire. Examples of the fiber include, for example, polyarylate fiber, aramid fiber, ultra-high molecular weight polyethylene fiber, PBO fiber, carbon fiber, and others. The fiber may be monofilament or multifilament.

An arrangement pattern of the wire in the reinforcing layer 13 is not particularly limited, and examples thereof include a spiral shape, a mesh shape, a braid shape, and others. Among them, it is preferable that the wire of the reinforcing layer 13 are arranged in a braided shape, thereby effectively increasing rigidity of the resulting tube.

The reinforcing layer 13 may be disposed only in a partial section of the first inner cylindrical body 11 or may be disposed in the entire section of the first inner cylindrical body 11 in the longitudinal direction. The reinforcing layer 13 may be disposed only in a partial section of the second inner cylindrical body 12 or may be disposed in the entire section of the second inner cylindrical body 12 in the longitudinal direction. At the boundary between the first inner cylindrical body 11 and the second inner cylindrical body 12, the reinforcing layer 13 is disposed so as to cross the boundary from the first inner cylindrical body 11 to the second inner cylindrical body 12.

The length of the reinforcing layer 13 in the longitudinal direction is not particularly limited, and the reinforcing layer 13 is preferably disposed on the outer side of the first inner cylindrical body 11 with a length of 10 mm or longer, more preferably 15 mm or longer, and is preferably disposed on the outer side of the second inner cylindrical body 12 with a length of 10 mm or longer, more preferably 15 mm or longer. As a result, the resulting tube becomes less likely to break at the boundary between the first inner cylindrical body 11 and the second inner cylindrical body 12.

For example, it is preferable that the length of a portion of the reinforcing layer 13 that is disposed on the outer side of the first inner cylindrical body 11 is longer than the length of a portion of the reinforcing layer 13 that is disposed on the outer side of the second inner cylindrical body 12, with respect to the longitudinal direction. Thereby, desired characteristics, derived from the first inner cylindrical body 11, can be imparted to the inner layer, that is disposed on the inner side of the reinforcing layer 13, over a wider range of the section where the reinforcing layer 13 is provided.

In the outer layer arrangement step, an outer layer 14 is disposed on the outer side of the reinforcing layer 13 to obtain a tube precursor 15. The outer layer 14 forms an outer layer of the tube. The outer layer 14 disposed on the outer side of the reinforcing layer 13 may have a cylindrical shape or a sheet shape. In the former case, the outer layer 14 of a cylindrical shape is disposed outside the reinforcing layer 13. In the latter case, it is preferable that the outer layer 14 of a sheet shape is wound around the outside of the reinforcing layer 13. The outer layer 14 may be composed of a heat-shrinkable film. In this case, the outer layer 14 is disposed outside the reinforcing layer 13 relatively loosely in the outer layer arrangement step, and the outer layer 14 is heated in the later heating step, whereby the outer layer 14 can be brought into close contact with the outer side of the reinforcing layer 13. The outer layer 14 can also be formed by applying resin to the outer side of the reinforcing layer 13. For example, the outer layer 14 may also be formed by dipping the first inner cylindrical body 11 and the second inner cylindrical body 12 with the reinforcing layer 13 disposed on the outside in a paint-like resin.

It is preferable that the outer layer 14 is disposed so as to cover the entire outer side of the reinforcing layer 13. The length of the outer layer 14 in the longitudinal direction may be longer than the length of the reinforcing layer 13 in the longitudinal direction. For example, the tube precursor 15 may have a section including the first inner cylindrical body 11 and the outer layer 14 but not including the reinforcing layer 13, or may have a section including the second inner cylindrical body 12 and the outer layer 14 but not including the reinforcing layer 13.

The outer layer 14 is made of resin. The type of resin constituting the outer layer 14 is not particularly limited, but the melting point of the resin constituting the outer layer 14 is lower than the melting point or decomposition point of the resin constituting the first inner cylindrical body 11. By forming the outer layer 14 from a resin with a relatively low melting point, it becomes easy to melt the outer layer 14 in the later heating step, whereby the second inner cylindrical body 12, the reinforcing layer 13 and the outer layer 14 can be easily integrated. The melting point of the resin constituting the outer layer 14 and the melting point of the resin constituting the second inner cylindrical body 12 are preferably close to each other, and the difference between the melting point of the resin constituting the outer layer 14 and the melting point of the resin constituting the second inner cylindrical body 12 is preferably 25° C. or lower, more preferably 20° C. or lower, and even more preferably 10° C. or lower.

For specific examples of the resin constituting the outer layer 14, the explanation of the resin constituting the first inner cylindrical body 11 and the second inner cylindrical body 12 above is referred. The outer layer 14 is preferably made of a polyester resin, a polyamide resin, a polyolefin resin, a polyurethane resin or a polyether polyamide resin. By forming the second inner cylindrical body 12 from such a resin, it becomes easy to melt and bond the second inner cylindrical body 12 and the outer layer 14 to each other in the later heating step.

The resin constituting the outer layer 14 may be the same as or different from the resin constituting the second inner cylindrical body 12. From the viewpoint of easily integrating the second inner cylindrical body 12, the reinforcing layer 13 and the outer layer 14, it is preferable that the resin constituting the outer layer 14 and the resin constituting the second inner cylindrical body 12 are the same.

In the case where the tube is applied to a catheter or a medical tubular object delivery device, the thickness of the outer layer 14 may be, for example, about 0.01 mm to 0.3 mm.

In the heating step, the tube precursor 15 is heated. By heating the tube precursor 15, a tube is obtained. The tube precursor 15 is heated by a heating means 16. Examples of the heating means 16 include a heater, a welding apparatus and the like. Heating of the tube precursor 15 may be performed, for example, by placing the tube precursor 15 in a heating furnace, or by applying hot air to the tube precursor 15, or the tube precursor 15 may be heated by ultrasonic welding or high frequency welding, which is performed by applying ultrasonic waves or high frequency waves to the tube precursor 15. In either case, a part of the second inner cylindrical body 12 and/or a part of the outer layer 14 may be melted and welded to each other. In the case where the tube precursor 15 is heated by a heating furnace or hot air, a heat shrinkable tube may be covered on the tube precursor 15 to heat it. In FIG. 1(D), the tube precursor 15 is heated while being moved relative to the heating means 16 as shown by a black arrow; however, the tube precursor 15 may be heated by the heating means 16 without being moved, or the tube precursor 15 may be heated while the heating means 16 is moved relative to the tube precursor 15.

Heating temperature in the heating step is equal to or higher than the melting point of the resin constituting the second inner cylindrical body 12 and the melting point of the resin constituting the outer layer 14, and lower than the melting point or decomposition point of the resin constituting the first inner cylindrical body 11. That is, in the heating step, a part of the second inner cylindrical body 12 and/or a part of the outer layer 14 is melted by heating, but the first inner cylindrical body 11 is not melted. By heating the tube precursor 15 in this manner, a part of the second inner cylindrical body 12 and/or a part of the outer layer 14 is melted and welded to each other, and the integrity of the second inner cylindrical body 12, the reinforcing layer 13 and the outer layer 14 can be enhanced. Meanwhile, the first inner cylindrical body 11 is supported from the outer surface and the side surface thereof by the outer layer 14 and the second inner cylindrical body 12 which are integrated. As a result, the obtained tube comes to have excellent integrity of the first inner cylindrical body 11, the second inner cylindrical body 12, the reinforcing layer 13 and the outer layer 14. It is preferable that the heating temperature in the heating step is higher than the melting point of the resin constituting the second inner cylindrical body 12 and the melting point of the resin constituting the outer layer 14.

As shown in FIG. 1, it is preferable that the tube precursor 15 has a section including the first inner cylindrical body 11, the reinforcing layer 13 and the outer layer 14, a section including the second inner cylindrical body 12, the reinforcing layer 13 and the outer layer 14, and a section including the second inner cylindrical body 12 and the outer layer 14 but not including the reinforcing layer 13, in this order from the first inner cylindrical body 11 to the second inner cylindrical body 12. That is, in the tube precursor 15, it is preferable that the section including the first inner cylindrical body 11, the reinforcing layer 13 and the outer layer 14, the section including the second inner cylindrical body 12, the reinforcing layer 13 and the outer layer 14, and the section including the second inner cylindrical body 12 and the outer layer 14 but not including the reinforcing layer 13 are arranged side by side in this order in the longitudinal direction. In the tube precursor 15 thus configured, the second inner cylindrical body 12 extends from the portion where it overlaps with the reinforcing layer 13 to the portion where it does not overlap with the reinforcing layer 13, and the outer layer 14 also extends from the portion where it overlaps with the reinforcing layer 13 to the portion where it does not overlap with the reinforcing layer 13. Therefore, by heating the tube precursor 15 in the heating step, the second inner cylindrical body 12 and the outer layer 14 can be joined to each other more firmly in the section where the reinforcing layer 13 is not present. In addition, a tube having the reinforcing layer 13 is normally prone to breakage or kinking due to unreasonable force caused by the difference in rigidity at a longitudinal end of the reinforcing layer 13; however, the tube obtained as described above is less likely to break or kink at the longitudinal end of the reinforcing layer 13 because the boundary between the first inner cylindrical body 11 and the second inner cylindrical body 12 does not overlap with the longitudinal end of the reinforcing layer 13. In the tube precursor 15, it is preferable that the section including the second inner cylindrical body 12 and the outer layer 14 but not including the reinforcing layer 13 is disposed at the end of the tube precursor 15 in the longitudinal direction, that is, it is formed to include the longitudinal end of the tube precursor 15.

In forming the tube precursor 15 as described above, It is preferable that the reinforcing layer 13 is disposed from the outer part of the first inner cylindrical body 11 to the outer part of the second inner cylindrical body 12 and is disposed in a partial section of the second inner cylindrical body 12 in the reinforcing layer arrangement step. At this time, it is preferable that the reinforcing layer 13 is not disposed at the end of the second inner cylindrical body 12 in the longitudinal direction opposite to the first inner cylindrical body 11. In the outer layer arrangement step, it is preferable that the outer layer 14 is disposed from the outer side of the reinforcing layer 13 to a section of the second inner cylindrical body 12 where the reinforcing layer 13 is not arranged. In FIG. 1(C), the outer layer 14 is disposed so as to be in contact with the outer part of the second inner cylindrical body 12 in the section of the second inner cylindrical body 12 where the reinforcing layer 13 is not arranged in the outer layer arrangement step; however, the outer layer 14 may be disposed apart from the outer side of the second inner cylindrical body 12 in that section.

Although not shown in FIG. 1, the tube precursor 15 may have a section including the first inner cylindrical body 11 and the outer layer 14 but not including the reinforcing layer 13. In this case, the reinforcing layer 13 is disposed from the outer part of the first inner cylindrical body 11 to the outer part of the second inner cylindrical body 12 and is disposed in a partial section of the first inner cylindrical body 11 in the reinforcing layer arrangement step. In the outer layer arrangement step, it is preferable that the outer layer 14 is disposed from the outer side of the reinforcing layer 13 to a section of the first inner cylindrical body 11 where the reinforcing layer 13 is not arranged.

Although not shown in FIG. 1, in the inner layer arrangement step, a plurality of at least one of the first inner cylindrical body 11 and the second inner cylindrical body 12 may be provided, and the first inner cylindrical body 11 and the second inner cylindrical body 12 may be disposed alternately in the longitudinal direction. In this case, it is preferable that the reinforcing layer 13 is disposed on the outer side of each of the plurality of first inner cylindrical body 11 and/or second inner cylindrical body 12.

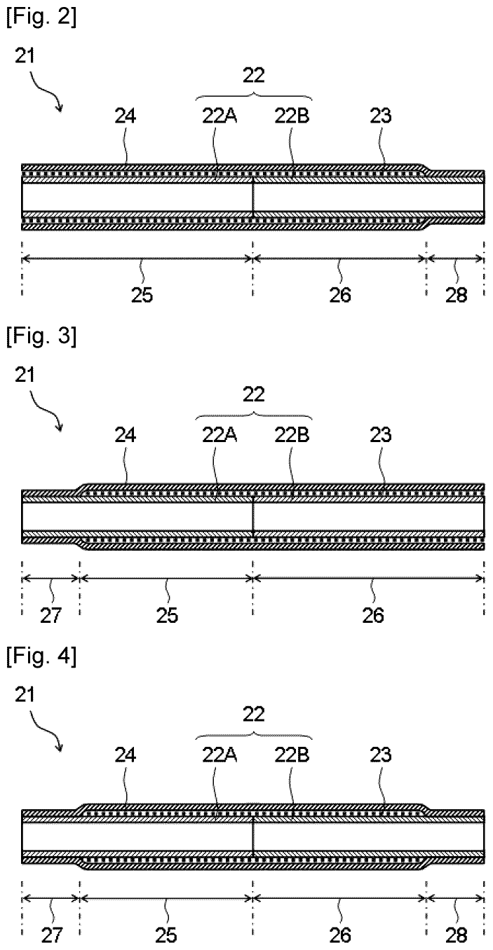

Next, a tube of the present invention is explained with reference to FIG. 2. FIG. 2 shows a cross-sectional view along the longitudinal direction of the tube obtained by the manufacturing method shown in FIG. 1. Note that the tube of the present invention is not limited to the embodiment shown in FIG. 2, nor is it limited to that obtained by the method for manufacturing the tube of the present invention.

A tube 21 according to the embodiment of the present invention extends in the longitudinal direction and comprises an inner layer 22, an outer layer 24 and a reinforcing layer 23 disposed therebetween, wherein the inner layer 22 includes a first inner layer 22A and a second inner layer 22B disposed side by side in the longitudinal direction, the reinforcing layer 23 is disposed from an outer side of the first inner layer 22A to an outer side of the second inner layer 22B, and a melting point or decomposition point of a resin constituting the first inner layer 22A is higher than a melting point of a resin constituting the second inner layer 22B and a melting point of a resin constituting an outer layer 24. The explanation of the first inner layer 22A, the second inner layer 22B, the reinforcing layer 23 and outer layer 24 in the tube 21 refers to the description of the first inner cylindrical body 11, the second inner cylindrical body 12, the reinforcing layer 13 and the outer layer 14 in the above method for manufacturing the tube, respectively.

By configuring the tube 21 as described above, desired characteristics, derived from the first inner layer 22A, can be imparted to the inner layer 22 disposed on the inner side of the reinforcing layer 23. Since the first inner layer 22A is made of a resin with a relatively high melting point or high decomposition point, the variety of resins that can be used for the first inner layer 22A increases, and it is easy to impart desired characteristics to the first inner layer 22A. For example, the first inner layer 22A can be made of a fluororesin, a polyimide resin, a polyamideimide resin or an aromatic polyetherketone resin, whereby slipperiness of the first inner layer 22A can be enhanced, and the strength of the first inner layer 22A can be increased. Therefore, it becomes easy to insert another object into a lumen of the tube 21, and an inner surface of the tube 21 is less likely to be damaged by another object inserted into the lumen of the tube 21.

In general, in a tube having a reinforcing layer, the integrity of the inner layer, the reinforcing layer and the outer layer can be increased by welding; whereas in the tube 21, since the first inner layer 22A is made of a resin with a relatively high melting point or high decomposition point, the first inner layer 22A is difficult to melt and bond. However, in the tube 21, since the second inner layer 22B is disposed adjacent to the first inner layer 22A in the longitudinal direction and the second inner layer 22B and the outer layer 24 are made of a relatively low melting point resin, the second inner layer 22B, the reinforcing layer 23 and the outer layer 24 can be integrated by welding. The first inner layer 22A is supported from the outer surface and the side surface of the first inner layer 22A by the outer layer 24 and the second inner layer 22B which are integrated, thereby stably held on the inner side of the tube 21. Therefore, in the tube 21, the integrity of the first inner layer 22A, the second inner layer 22B, the reinforcing layer 23 and the outer layer 24 comes to be ensured.

Furthermore, in the tube 21, since the second inner layer 22B and outer layer 24 are made of a relatively low melting point resin, it becomes easy to join to another tube by welding. For example, it becomes easier to insert another tube into the tube 21 through an opening on the second inner layer 22B side and weld an outer surface of the another tube to the second inner layer 22B of the tube 21, or to insert the tube 21 to another tube and weld an inner surface of the another tube to the outer layer 24 of the tube 21.

The second inner layer 22B and the outer layer 24 are preferably made of, for example, a polyester resin, a polyamide resin, a polyolefin resin, a polyurethane resin or a polyether polyamide resin, as a resin with excellent weldability.

In addition, in the tube 21, the boundary between the first inner layer 22A and the second inner layer 22B is located at a position where it overlaps with the reinforcing layer 23, so that the boundary between the first inner layer 22A and the second inner layer 22B is reinforced by the reinforcing layer 23, and the tube 21 is less likely to break at the boundary between the first inner layer 22A and the second inner layer 22B when using the tube 21. Therefore, the reliability of the tube 21 can be improved.

As shown in FIG. 2, it is preferable that the tube 21 has a section 25 including the first inner layer 22A, the reinforcing layer 23 and the outer layer 24, a section 26 including the second inner layer 22B, the reinforcing layer 23 and the outer layer 24, and a section 28 including the second inner layer 22B and the outer layer 24 but not including the reinforcing layer 23, in this order. That is, in the tube 21, it is preferable that the section 25 including the first inner layer 22A, the reinforcing layer 23 and the outer layer 24, the section 26 including the second inner layer 22B, the reinforcing layer 23 and the outer layer 24, and the section 28 including the second inner layer 22B and the outer layer 24 but not including the reinforcing layer 23 are arranged side by side in this order in the longitudinal direction. In the tube 21 configured in this manner, the boundary between the first inner layer 22A and the second inner layer 22B does not overlap with a longitudinal end of the reinforcing layer 23, and the tube 21 is less prone to breakage or kinking at the end of the reinforcing layer 23. Therefore, the reliability of the tube 21 can be improved. In the tube 21, it is preferable that the section 28 including the second inner layer 22B and the outer layer 24 but not including the reinforcing layer 23 is disposed at the end of the tube 21 in the longitudinal direction, that is, it is formed to include the longitudinal end of the tube 21.

FIGS. 3 and 4 show other configuration examples of the tube 21. In the tube 21, the reinforcing layer 23 may be disposed only in a partial section of the first inner layer 22A in the longitudinal direction in the portion where it overlaps with the first inner layer 22A. For example, as shown in FIG. 3, the tube 21 may have a section 27 including the first inner layer 22A and the outer layer 24 but not including the reinforcing layer 23. In addition, as shown in FIG. 4, the tube 21 may have the section 27 including the first inner layer 22A and the outer layer 24 but not including the reinforcing layer 23, and the section 28 including the second inner layer 22B and the outer layer 24 but not including the reinforcing layer 23. In the section 28, since the second inner layer 22B and the outer layer 24 can be joined to each other more firmly by welding, the tube 21 preferably has the section 28 including the second inner layer 22B and the outer layer 24 but not including the reinforcing layer 23, as shown in FIGS. 2 and 4. As a result, the integrity of the tube 21 can be improved.

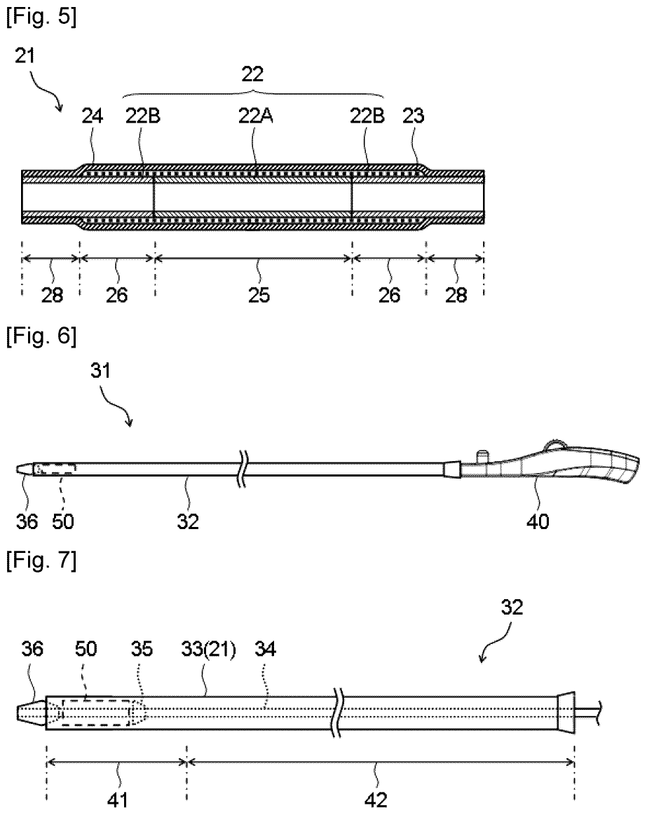

FIG. 5 shows still another configuration example of the tube 21. In the tube 21, a plurality of at least one of the first inner layer 22A and the second inner layer 22B may be provided, and the first inner layer 22A and the second inner layer 22B may be disposed alternately in the longitudinal direction. For example, as shown in FIG. 5, in the tube 21, the second inner layers 22B may be disposed on both sides of the first inner layer 22A in the longitudinal direction. In this case, it is preferable that the reinforcing layer 23 is disposed from the outer side of the first inner layer 22A to the outer side of the second inner layers 22B provided on both sides of the first inner layer 22A in the longitudinal direction. That is, the reinforcing layer 23 preferably extends from the outer side of the second inner layer 22B provided on one side of the first inner layer 22A in the longitudinal direction to the outer side of the second inner layer 22B provided on the other side of the first inner layer 22A in the longitudinal direction. In the tube 21, it is preferable that the second inner layers 22B having a relatively low melting point, provided on both sides of the first inner layer 22A in the longitudinal direction, are joined by welding to the outer layer 24 having a relatively low melting point, disposed on the outer sides of the first inner layer 22A and the second inner layer 22B. As a result, the first inner layer 22A is supported from both sides in the longitudinal direction and the outer surface thereof by the outer layer 24 and the second inner layer 22B which are integrated, and the first inner layer 22A can be held more stably on the inner side of the tube 21.

In the embodiment shown in FIG. 5, it is preferable that the section 28 including the second inner layer 22B and the outer layer 24 and not including the reinforcing layer 23 is provided on one or both sides of the second inner layers 22B disposed on both sides of the first inner layer 22A in the longitudinal direction. Thereby, the integrity of the tube 21 can be further improved. More preferably, as shown in FIG. 5, the sections 28 including the second inner layer 22B and the outer layer 24 and not including the reinforcing layer 23 are provided on both sides of the second inner layers 22B disposed on both sides of the first inner layer 22A in the longitudinal direction.

The tube of the present invention can be suitably used for a component of a catheter. According to the tube of the present invention, it becomes easy to insert another object into the lumen and to move forward or backward the another object in the lumen, or to slide the tube against another object with the another object placed in the lumen. Alternatively, the inner surface of the tube can be made less susceptible to damage by another object inserted into the lumen. Furthermore, due to the reinforcing layer, it has high breaking strength. Therefore, by applying the tube of the present invention to a catheter, a highly reliable catheter can be obtained.

A catheter comprises a shaft and a control part provided on a proximal side of the shaft, and the tube of the present invention can be used for the shaft. In the case of treatment using an endoscope, the shaft is inserted into an instrument channel through a channel port of the endoscope to reach a lesion site and treatment or the like of the lesion site is performed.

Examples of the catheter include a guiding catheter, an electrode catheter, an ablation catheter, a balloon catheter, a mapping catheter, a microcatheter, a stent delivery system (a medical tubular object delivery device), and the like. In this case, examples of another object inserted or placed in the tube include a guide wire, a treatment instrument, a stent, and the like.

The tube of the present invention can be particularly suitably used in a medical tubular object delivery device. A medical tubular object delivery device is a device for delivering a medical tubular object into the body, and a medical tubular object such as a stent is placed in a lumen of the shaft. By delivering a medical tubular object to a lesion site using the medical tubular object delivery device and placing it in a body cavity, various diseases caused by narrowing or obstruction of a body cavity, such as the bile duct, other digestive tracts and blood vessels, can be treated. At this time, when the tube of the present invention is used for the shaft of the medical tubular object delivery device, it becomes easier to stably implant a medical tubular object in the body.

A medical tubular object delivery device according to an embodiment of the present invention is explained with reference to FIGS. 6 to 10. FIG. 6 shows an overall schematic view of a medical tubular object delivery device according to an embodiment of the present invention, and FIGS. 7 to 10 show configuration examples of a shaft of the medical tubular object delivery device. However, the medical tubular object delivery device of the present invention is not limited to the embodiments shown in FIGS. 6 to 10.

A medical tubular object delivery device 31 is a long device comprising a shaft 32, and a medical tubular object 50 is placed in a lumen of the shaft 32. The length of the shaft 32 in the longitudinal direction may be, for example, about 800 mm to 3000 mm. In the following description, the medical tubular object delivery device may be simply referred to as a “delivery device”.

The shaft 32 extends in the longitudinal direction, and has a proximal side and a distal side with respect to the longitudinal direction. In the delivery device 31, the proximal side refers to a user's hand side, and the distal side refers to the opposite direction to the proximal side, that is, a direction toward a treatment target side. In FIGS. 6 to 10, the right side of the drawings corresponds to the proximal side, and the left side of the drawings corresponds to the distal side.

It is preferable that a control part 40 is provided on the proximal side of the shaft 32. By manipulating the control part 40, the medical tubular object 50 can be exposed to the outside of the shaft 32 in the patient's body, and the medical tubular object 50 can be left in the patient's body.

In the delivery device 31, a distal portion of the shaft 32 can be composed from the tube 21 of the present invention, and the medical tubular object 50 can be placed in the lumen of the tube 21. Hereinafter, the tube of the present invention provided at the distal portion of the shaft 32 may be referred to as an “outer tube”.

A typical example of the medical tubular object 50 is a stent. The medical tubular object 50 may be a coiled medical tubular object formed from a single linear metal or polymeric material, a medical tubular object formed by cutting out a metal tube or a polymeric material tube using a laser or the like, a medical tubular object formed by assembling and welding linear members, or a medical tubular object formed by weaving linear metals. Examples of the medical tubular object 50 include stent grafts, obturators, injection catheters, prosthetic valves and others, in addition to stents.

From the viewpoint of an expansion mechanism, the medical tubular object 50 can be classified into (i) a balloon-expanding medical tubular object, that is delivered to a lesion site in the state where the medical tubular object is mounted on the surface of a balloon and is expanded by the balloon at the lesion site, and (ii) a self-expanding medical tubular object, that is delivered to a lesion site in the state where the expansion is suppressed and expands itself at the lesion site by removing a member that suppresses the expansion. The delivery device 31 is suitably used to deliver a self-expanding medical tubular object, and the outer tube 33 functions as a member that suppresses expansion of the medical tubular object 50. The medical tubular object 50 contracts in the radial direction and extends in the longitudinal direction in the state of being placed in the lumen of the outer tube 33, resulting in a reduced diameter state, which is a more elongated cylindrical form than the expanded state. After delivering the medical tubular object 50 to a lesion site, the outer tube 33 is slid proximally with respect to the medical tubular object 50, whereby the medical tubular object 50 is exposed to the outside of the shaft 32, and the medical tubular object 50 expands in the radial direction and is left in the body.

It is preferable that the outer tube 33 has a first section 41 including a distal end thereof and a second section 42 proximal to the first section 41 in the longitudinal direction, the first inner layer 22A is provided at the first section 41, the second inner layer 22B is provided at the second section 42, and the medical tubular object 50 is placed in a lumen of the first section 41 of the outer tube 33. By placing the medical tubular object 50 in the lumen of the first section 41 of the outer tube 33, it becomes easier to perform the operation of sliding to the outer tube 33 proximally relative to the medical tubular 50 stably when the medical tubular object 50 is left in the body. In the delivery device 31, in sliding the outer tube 33 proximally relative to the medical tubular object 50, a large frictional resistance tends to occur between the outer tube 33 and the medical tubular object 50. At this time, by forming the inner layer of the first section 41 of the outer tube 33 from the first inner layer 22A, the frictional resistance between the outer tube 33 and the medical tubular object 50 can be reduced. Or, the inner surface of the outer tube 33 is less likely to be damaged by the frictional resistance with the medical tubular object 50, and the medical tubular object 50 is less likely to get caught on the inner surface of the outer tube 33. Therefore, the medical tubular object 50 can be stably expanded.

The inner diameter of the outer tube 33 is appropriately determined according to the outer diameter of the medical tubular object 50 (the outer diameter of the medical tubular object 50 in a reduced diameter state) to be inserted, and is, for example, about 0.3 mm to 3.5 mm.

The length of the outer tube 33 in the longitudinal direction is preferably 1.2 times or more, more preferably 1.5 times or more the length of the medical tubular object 50 of the reduced diameter state in the longitudinal direction. The upper limit of the length of the outer tube 33 in the longitudinal direction is not particularly limited, and may be, for example, 3000 mm or shorter, 2000 mm or shorter, 1000 mm or shorter, or 500 mm or shorter. The length of the first section 41 of the outer tube 33 in the longitudinal direction is preferably longer than the length of the medical tubular object 50 of the reduced diameter state in the longitudinal direction. The length of the first section 41 of the outer tube 33 is, for example, preferably 1.1 times or more, more preferably 1.2 times or more the length of the medical tubular object 50 of the reduced diameter state in the longitudinal direction. The upper limit of the length of the first section 41 of the outer tube 33 in the longitudinal direction is not particularly limited. The length of the second section 42 of the outer tube 33 in the longitudinal direction is not particularly limited, and the second section 42 can be a section located proximal to the first section 41 in the outer tube 33.

It is preferable that the shaft 32 comprises the outer tube 33 and an inner tube 34 disposed in the lumen of the outer tube 33, and the medical tubular object 50 is disposed between the outer tube 33 and the inner tube 34. Thereby, the medical tubular object 50 can be stably held in the lumen of the outer tube 33. Furthermore, when the medical tubular object 50 is left in the body, it becomes easier to stably perform the operation of pulling the outer tube 33 proximally to expose the medical tubular object 50.

The inner tube 34 is disposed at least at the distal portion of the shaft 32 and may extend to a proximal portion of the shaft 32. A lumen of the inner tube 34 can function as a guidewire passage. In the case where the delivery device 31 is an over-the-wire type one, the inner tube 34 preferably extends from the distal portion of the shaft 32 to the proximal portion of the shaft 32, and in the case where the delivery device 31 is a rapid exchange type one, the inner tube 34 preferably extends from the distal portion of the shaft 32 halfway to the proximal portion of the shaft 32.

The inner tube 34 is preferably provided with a stopper 35 on the proximal side of the medical tubular object 50. The stopper 35 is attached to the outer surface of the inner tube 34. The stopper 35 is disposed in contact with or near a proximal end of the medical tubular object 50. By providing the stopper 35 on the proximal side of the medical tubular object 50, when pulling the outer tube 33 proximally, the stopper 35 comes into contact with the proximal end of the medical tubular object 50, which prevents the medical tubular object 50 from being pulled proximally with the outer tube 33. Thereby, it becomes easier to expose the medical tubular object 50 to the outside of the shaft 32. For example, It is preferable that the stopper 35 is arranged so that the part of the stopper 35 that comes into contact with the proximal end of the medical tubular object 50 when the outer tube 33 is pulled proximally is positioned within 10 mm proximally from the proximal end of the medical tubular object 50.

The shape of the stopper 35 can be, for example, a ring shape. It is preferable that the outer diameter of the stopper 35 is smaller than or equal to the inner diameter of the outer tube 33 and larger than or equal to the inner diameter of the medical tubular object 50 placed in the lumen of the outer tube 33. The stopper 35 can be made of resin, metal, or a composite material thereof. In particular, the stopper 35 is preferably made of elastomer resin, thereby preventing deformation of or damage to the medical tubular object 50 when the stopper 35 comes into contact with the medical tubular object 50. As the elastomer resin, a polyamide resin is preferably used, whereby the rigidity of the stopper 35 is increased and the proximal end of the medical tubular object 50 is supported by the stopper 35, resulting in enabling the medical tubular object 50 to be effectively deployed.

A distal tip 36 is preferably provided at a distal end of the inner tube 34. The distal tip 36 preferably has a lumen that communicates with the lumen of the inner tube 34. The distal tip 36 forms a distal end portion of the shaft 32, and prevents a distal end of the shaft 32 from damaging a body cavity when the shaft 32 is inserted into an instrument channel of an endoscope to deliver the medical tubular object 50 to a lesion site. In addition, followability of the shaft 32 to the preceding guide wire or instrument channel and deliverability of the distal end of the shaft 32 to the lesion site can be improved, and operability of the delivery device 31 can be improved.

The distal tip 36 is preferably made of elastomer resin. Preferred examples of the elastomer resin include polyurethane resins, polyester resins, polyamide resins and others, and it is particularly preferred to be composed of a polyamide resin. By configuring the distal tip 36 in this manner, followability of the distal tip 36 to the guide wire and safety of the distal end of the shaft 32 can be improved.

The shaft 32 may be provided with an X-ray opaque marker. By providing the X-ray opaque marker on the shaft 32, the position of the shaft 32 in the body can be confirmed under X-ray fluoroscopy using the X-ray opaque marker as a mark. The X-ray opaque marker is preferably provided near the portion of the shaft 32 where the medical tubular object 50 is placed, and is preferably provided on the distal tip 36, the stopper 35 or the outer tube 33. By providing the X-ray opaque marker on the distal tip 36, the position of the distal end of the shaft 32 can be confirmed under X-ray fluoroscopy. By providing the X-ray opaque marker on the stopper 35, the position and extruded state of the medical tubular object 50 can be confirmed under X-ray fluoroscopy. In the case where the X-ray opaque marker is provided on the outer tube 33, the timing at which the medical tubular object 50 is exposed from the outer tube 33 can be grasped under X-ray fluoroscopy. The number of X-ray opaque markers installed may be one or may be multiple.

The shaft 32 may comprise a tubular member other than the outer tube 33 and the inner tube 34. For example, a traction tube can be provided so as to be connected to a proximal end portion of the outer tube 33, or a protection tube can be provided outside the outer tube 33 or the traction tube.

The configurations of the shaft 32 shown in FIGS. 7 to 10 are specifically explained. The shaft 32 shown in FIG. 7 comprises an outer tube 33 and an inner tube 34 disposed in the lumen of the outer tube 33. A medical tubular object 50 with a reduced diameter is provided at a distal portion of the shaft 32 between the outer tube 33 and the inner tube 34. The outer tube 33 is the tube 21 of the present invention.

The inner tube 34 extends from the distal portion of the shaft 32 to a proximal portion of the shaft 32, that provides a delivery device 31 of the over-the-wire type. The outer tube 33 extends from the distal portion of the shaft 32 to the proximal portion of the shaft 32, and the inner tube 34 extends proximally beyond a proximal end of the outer tube 33. A distal tip 36 is provided at a distal end of the inner tube 34, and a stopper 35 is provided on the outer surface of the inner tube 34 on the proximal side of the medical tubular object 50. The outer tube 33 is slidable in the longitudinal direction relative to the inner tube 34. In the delivery device 31 equipped with the shaft 32 shown in FIG. 7, when the outer tube 33 is pulled proximally, the medical tubular object 50 is exposed outside the shaft 32, and the medical tubular object 50 can be left in the body.

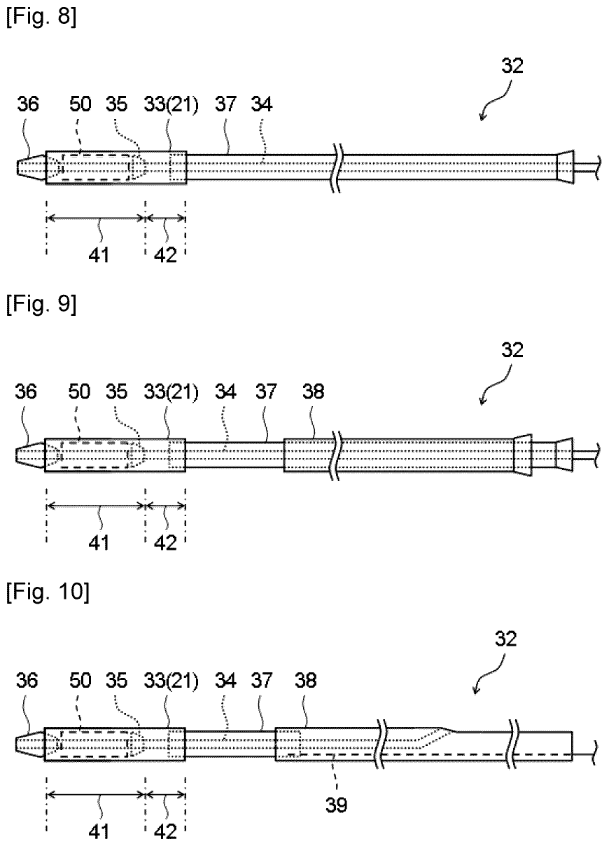

The shaft 32 shown in FIG. 8 comprises an outer tube 33, a traction tube 37 connected to a proximal portion of the outer tube 33, and an inner tube 34 disposed in the lumen of the outer tube 33 and the lumen of the traction tube 37. A medical tubular object 50 is provided at a distal portion of the shaft 32 between the outer tube 33 and the inner tube 34. The outer tube 33 is the tube 21 of the present invention.

The inner tube 34 extends from the distal portion of the shaft 32 to a proximal portion of the shaft 32, that provides a delivery device 31 of the over-the-wire type. The outer tube 33 is disposed at the distal portion of shaft 32. The traction tube 37 connected to the proximal portion of the outer tube 33 extends proximally beyond a proximal end of the outer tube 33 and extends to the proximal portion of the shaft 32. The inner tube 34 extends proximally beyond a proximal end of traction tube 37. A distal tip 36 is provided at a distal end of the inner tube 34, and a stopper 35 is provided on the outer surface of the inner tube 34 on the proximal side of the medical tubular object 50. The outer tube 33 and the traction tube 37 are slidable in the longitudinal direction relative to the inner tube 34. In the delivery device 31 equipped with the shaft 32 shown in FIG. 8, when the traction tube 37 is pulled proximally, the outer tube 33 moves proximally, the medical tubular object 50 is exposed outside the shaft 32, and the medical tubular object 50 can be left in the body.

The shaft 32 shown in FIG. 9 comprises an outer tube 33, a traction tube 37 connected to a proximal portion of the outer tube 33, an inner tube 34 disposed in the lumen of the outer tube 33 and the lumen of the traction tube 37, and a protection tube 38 disposed outside the traction tube 37. A medical tubular object 50 with a reduced diameter is provided at a distal portion of the shaft 32 between the outer tube 33 and the inner tube 34. The outer tube 33 is the tube 21 of the present invention.

The inner tube 34 extends from the distal portion of the shaft 32 to a proximal portion of the shaft 32, that provides a delivery device 31 of the over-the-wire type. The outer tube 33 is disposed only at the distal portion of the shaft 32. The traction tube 37 connected to the proximal portion of the outer tube 33 extends proximally beyond a proximal end of the outer tube 33 and extends to the proximal portion of the shaft 32. The inner tube 34 extends proximally beyond a proximal end of the traction tube 37. The protection tube 38 is disposed so as to cover only a portion of the traction tube 37, and the traction tube 37 extends distally beyond a distal end of the protection tube 38 and extends proximally beyond a proximal end of the protection tube 38. A distal tip 36 is provided at a distal end of the inner tube 34, and a stopper 35 is provided on the outer surface of the inner tube 34 on the proximal side of the medical tubular object 50. The outer tube 33 and the traction tube 37 are slidable in the longitudinal direction relative to the protection tube 38 and the inner tube 34. In the delivery device 31 equipped with the shaft 32 shown in FIG. 9, when the traction tube 37 is pulled proximally, the outer tube 33 moves proximally, the medical tubular object 50 is exposed outside the shaft 32, and the medical tubular object 50 can be left in the body.

The shaft 32 shown in FIG. 10 comprises an outer tube 33, a traction tube 37 connected to a proximal portion of the outer tube 33, a protection tube 38 disposed outside the traction tube 37 and extends proximally beyond a proximal end of the traction tube 37, an inner tube 34 disposed in the lumen of the outer tube 33, the lumen of the traction tube 37 and the lumen of the protection tube 38, and a linear traction member 39 attached to a proximal portion of the traction tube 37 and disposed in the lumen of the protection tube 38. A medical tubular object 50 with a reduced diameter is provided at a distal portion of the shaft 32 between the outer tube 33 and the inner tube 34. The outer tube 33 is the tube 21 of the present invention.

The inner tube 34 extends from the distal portion of the shaft 32 halfway to a proximal portion of the shaft 32, that provides a delivery device 31 of the rapid exchange type. By configuring the inner tube 34 to extend from the distal portion of the shaft 32 to the proximal portion of the shaft 32, a delivery device 31 of the over-the-wire type can be provided. The outer tube 33 is disposed only at the distal portion of the shaft 32, and the traction tube 37 connected to the proximal portion of the outer tube 33 extends proximally beyond a proximal end of the outer tube 33 halfway to the proximal portion of the shaft 32. The linear traction member 39 attached to a proximal portion of the traction tube 37 extends proximally beyond a proximal end of the traction tube 37 to the proximal portion of the shaft 32. The protection tube 38 is disposed so as to cover a part of the traction tube 37 and a part of the linear traction member 39, and the linear traction member 39 extends proximally beyond a proximal end of the protection tube 38. A distal tip 36 is provided at a distal end of the inner tube 34, and a stopper 35 is provided on the outer surface of the inner tube 34 on the proximal side of the medical tubular object 50. The outer tube 33 and the traction tube 37 are slidable in the longitudinal direction relative to the protection tube 38 and the inner tube 34. The linear traction member 39 is not fixed to the protection tube 38. In the delivery device 31 equipped with the shaft 32 shown in FIG. 10, when the linear traction member 39 is pulled proximally, the traction tube 37 and the outer tube 33 moves proximally, the medical tubular object 50 is exposed outside the shaft 32, and the medical tubular object 50 can be left in the body.

In the shafts 32 shown in FIGS. 9 and 10, when the traction tube 37 is pulled proximally for implanting the medical tubular object 50 in the body, a part of the portion of the traction tube 37 that was not covered by the protection tube 38 is housed in the lumen of the protection tube 38. As a result, when pulling the traction tube 37 proximally, it is possible to reduce the frictional resistance caused by the traction tube 37 coming into contact with an instrument channel or a channel port of an endoscope. Therefore, the operating load when pulling the outer tube 33 and the traction tube 37 proximally is reduced, and the medical tubular object 50 can be stably expanded. Furthermore, the entire shaft 32 can be restrained from moving proximally when pulling the outer tube 33 and the traction tube 37 proximally, and the medical tubular object 50 can be left in a desired position with high precision.

The inner tube 34, the traction tube 37 and the protection tube 38 are preferably made of resin. Examples of resin constituting these tubes include synthetic resins such as polyester resin (e.g., polyethylene terephthalate), polyamide resin (e.g., nylon), polyolefin resin (e.g., polyethylene and polypropylene), aromatic polyether ketone resin (e.g., PEEK), polyurethane resin, polyether polyamide resin, polyimide resin, polyamideimide resin, fluororesin (e.g., PTFE, PFA and ETFE), polyvinyl chloride resin, silicone resin, and others. Each tube may have a reinforcing layer made of wire.

The linear traction member 39 can be constructed from a thread made of metal wire or synthetic resin. The linear traction member 39 may be a composite body made of a plurality of materials, for example, a composite body of metal and synthetic resin. The linear traction member 39 may be configured so that a metal wire is coated with resin.

This application claims priority to Japanese Patent Application No. 2022-042890 filed on Mar. 17, 2022. All of the contents of the Japanese Patent Application No. 2022-042890 filed on Mar. 17, 2022 is incorporated by reference herein.

### REFERENCE SIGNS LIST

11: first inner cylindrical body
12: second inner cylindrical body
13: reinforcing layer
14: outer layer
15: tube precursor
16: heating means
21: tube
22: inner layer, 22A: first inner layer, 22B: second inner layer
23: reinforcing layer
24: outer layer
25: section including a first inner layer, a reinforcing layer and an outer layer
26: section including a second inner layer, a reinforcing layer and an outer layer
27: section including a first inner layer and an outer layer but not including a reinforcing layer
28: section including a second inner layer and an outer layer but not including a reinforcing layer
31: medical tubular object delivery device
32: shaft
33: outer tube
34: inner tube
35: stopper
36: distal tip
37: traction tube
38: protection tube
39: linear traction member
40: control part
41: first section
42: second section
50: medical tubular object

1. A method for manufacturing a tube extending in a longitudinal direction and having a reinforcing layer composed of wire, comprising the steps of:
disposing a first inner cylindrical body and a second inner cylindrical body side by side in the longitudinal direction;
disposing a reinforcing layer composed of wire from an outer side of the first inner cylindrical body to an outer side of the second inner cylindrical body;
disposing an outer layer on an outer side of the reinforcing layer to obtain a tube precursor; and
heating the tube precursor;
wherein:
a melting point or decomposition point of a resin constituting the first inner cylindrical body is higher than a melting point of a resin constituting the second inner cylindrical body and a melting point of a resin constituting the outer layer; and
a heating temperature in the step of heating the tube precursor is equal to or higher than the melting point of the resin constituting the second inner cylindrical body and the melting point of the resin constituting the outer layer, and lower than the melting point or decomposition point of the resin constituting the first inner cylindrical body.

2. The method for manufacturing a tube according to claim 1, wherein the first inner cylindrical body is made of a fluororesin, a polyimide resin, a polyamideimide resin or an aromatic polyether ketone resin.

3. The method for manufacturing a tube according to claim 1 or 2, wherein the second inner cylindrical body and the outer layer are each independently made of a polyester resin, a polyamide resin, a polyolefin resin, a polyurethane resin or a polyether polyamide resin.

4. The method for manufacturing a tube according to claim 1 or 2, wherein the second inner cylindrical body and the outer layer are made of a same resin.

5. The method for manufacturing a tube according to claim 1 or 2, wherein the tube precursor has a section including the first inner cylindrical body, the reinforcing layer and the outer layer, a section including the second inner cylindrical body, the reinforcing layer and the outer layer, and a section including the second inner cylindrical body and the outer layer but not including the reinforcing layer, in this order.

6. A tube extending in a longitudinal direction, comprising an inner layer, an outer layer and a reinforcing layer composed of wire disposed between the inner layer and the outer layer, wherein:
the inner layer includes a first inner layer and a second inner layer disposed side by side in the longitudinal direction;
the reinforcing layer is disposed from an outer side of the first inner layer to an outer side of the second inner layer; and
a melting point or decomposition point of a resin constituting the first inner layer is higher than a melting point of a resin constituting the second inner layer and a melting point of a resin constituting an outer layer.

7. The tube according to claim 6, wherein the first inner layer is made of a fluororesin, a polyimide resin, a polyamideimide resin or an aromatic polyether ketone resin.

8. The tube according to claim 6 or 7, wherein the second inner layer and the outer layer are each independently made of a polyester resin, a polyamide resin, a polyolefin resin, a polyurethane resin or a polyether polyamide resin.

9. The tube according to claim 6, wherein the second inner layer and the outer layer are made of a same resin.

10. The tube according to claim 6, wherein the tube has a section including the first inner layer, the reinforcing layer and the outer layer, a section including the second inner layer, the reinforcing layer and the outer layer, and a section including the second inner layer and the outer layer but not including the reinforcing layer, in this order.

11. A catheter comprising the tube according to claim 6, 7, 9 or 10.

12. A medical tubular object delivery device comprising the tube according to claim 6, 7, 9 or 10, wherein:
the tube has a first section including a distal end thereof and a second section proximal to the first section in the longitudinal direction;
the first inner layer is provided at the first section and the second inner layer is provided at the second section; and
a medical tubular object is placed in a lumen of the first section of the tube.
